(19) United States
(12) Patent Application Publication
(10) Pub. No.: US 2007/0288267 A1
(43) Pub. Date: Dec. 13, 2007

(54) Method of authentication using universally unique identifiers

(76) Inventor: Augustine Fou, New York, NY (US)

Correspondence Address: CHARLES A. RATTNER, 12 HOMEWOOD LANE, DARRIEN, CT, 06820-6109, US

(21) Appl. No.: 11/891,559

(22) Filed: Aug. 10, 2007

Related U.S. Application Data
(63) Continuation-in-part of application No. 10/653,391, filed on Sep. 2, 2003.
(60) Provisional application No. 60/837,398, filed on Aug. 10, 2006.

(51) Int. Cl. G06Q 30/00

(52) U.S. Cl. 705/2; 705/28

(57) ABSTRACT

Individual products may have affixed therto, or to its packaging, a universally unique item identifier that includes encoded information, or other unique code, as well as a specific time at which the code was generated and/or applied. The codes, as applied to the products and purchased by consumers, may be entered by consumers and the like into a publicly available computer system, such as a web site, to confirm the validity of the product. Counterfeit products are readily identified since the codes can never be re-used.

### CROSS-REFERENCE TO RELATED APPLICATIONS

This application claims priority under 35 U.S.C. §119 to U.S. Provisional Patent Application Ser. No. 60/837,398 entitled “METHOD OF AUTHENTICATION” filed in the name of Augustine Fou on Aug. 10, 2006, the entirety of which is herein incorporated by reference. This application further claims priority under 35 U.S.C. §120 as a continuation-in-part of co-pending U.S. patent application Ser. No. 10/653,391 entitled “METHOD FOR FACILITATING COMMERCIAL PROGRAMS INVOLVING CONSUMER PRODUCTS USING UNIVERSALLY UNIQUE ITEM IDENTIFIERS” filed in the name of Augustine Fou on Sep. 2, 2003, the entirety of which is herein incorporated by reference.

### TECHNICAL FIELD

This disclosure generally relates to automated electrical business practices, and in particular it relates to distribution and submission of codes for countering counterfeiting of products.

### BACKGROUND OF THE DISCLOSURE

Currently, it is well known that counterfeit drugs are entering the prescription drug supply at alarming rates, leading to massive losses and liability of lawsuits against pharmaceutical companies, skyrocketing insurance claims, and public health costs. New laws proposed to help stem this tide, such as the Prescription Drug Marketing Act that has “electronic pedigree” requirements for tracking pharmaceutical chains of custody, are deemed to be too costly and impractical to implement.

Some pharmaceutical companies are experimenting with putting RFID (radio-frequency identifiers) on units of prescription drugs. However, this is costly on a per unit basis, requires specialized RFID readers, and requires pharmacists to take time out to spot check drugs in order to be effective.

Accordingly, there is a need for a method for authenticating products, such as pharmaceuticals, which is easily implemented and effective to identify counterfeit products to consumers, product manufacturers, distributors and law enforcement authorities.

### BRIEF SUMMARY OF THE DISCLOSURE

According to the methods of authentication now disclosed, human-readable alphanumeric codes are printed on prescription drug labels to uniquely identify each unit. This allows consumers to verify the authenticity of the drugs simply by entering in the code into, for example, the manufacturer's or other third-party website. When a counterfeit item is detected, consumers can immediately return it to their pharmacy and report it to the manufacturer for further investigation. The operator of the website may take further actions upon the detection of a counterfeit product.

### BRIEF DESCRIPTION OF THE DRAWINGS

Further aspects of the present disclosure will be more readily appreciated upon review of the detailed description of its various embodiments, described below, when taken in conjunction with the accompanying drawing, of which:

FIG. 1 is a flowchart depicting an exemplary process of using Universally Unique Item Identifiers (UUIIs) to confirm the authenticity of a product, according to the present disclosure.

### DETAILED DESCRIPTION OF THE EMBODIMENTS

The present disclosure builds upon the methods for using universally unique item identifiers (UUIIs), as described in co-pending U.S. patent application Ser. No. 10/653,391, the entirety of which is incorporated herein by reference. UUIIs differ from known uniform product identifiers, such as stock keeping unit (SKU), uniform product codes (UPCs) and product serial numbers, in that UUIIs may include encoded information, such as a pseudo-random number applied to a particular product, or any other desired information.

UUIIs may be generated and supplied to third parties, such as manufacturers of products, for affixing to items in order to individually identify each item. The information in the UUII may be used by businesses to analyze and monitor the distribution of their product in the marketplace. A benefit of UUIIs to purchasers, consumers, and users of consumer products is the ease and efficiency of participating in confirming the validity of purchased products and identifying products that have been counterfeited (e.g., a UUII on a product is determined to be false).

By printing human readable UUII codes on units of pharmaceuticals, for example, to uniquely identify each item, consumers, manufacturers and distributors can easily check the authenticity of the drugs for themselves by visiting the appropriate pharmaceutical company's website, or other globally-accessible network site, and entering the code. Consumers should be willing to spend the few minutes each to ensure their family member is not unknowingly taking counterfeit drugs. With potentially millions of consumers helping to check for and report such counterfeit drugs, the speed of detection of counterfeit products is greatly increased, and the entire pharmaceutical supply may thus be rendered much safer.

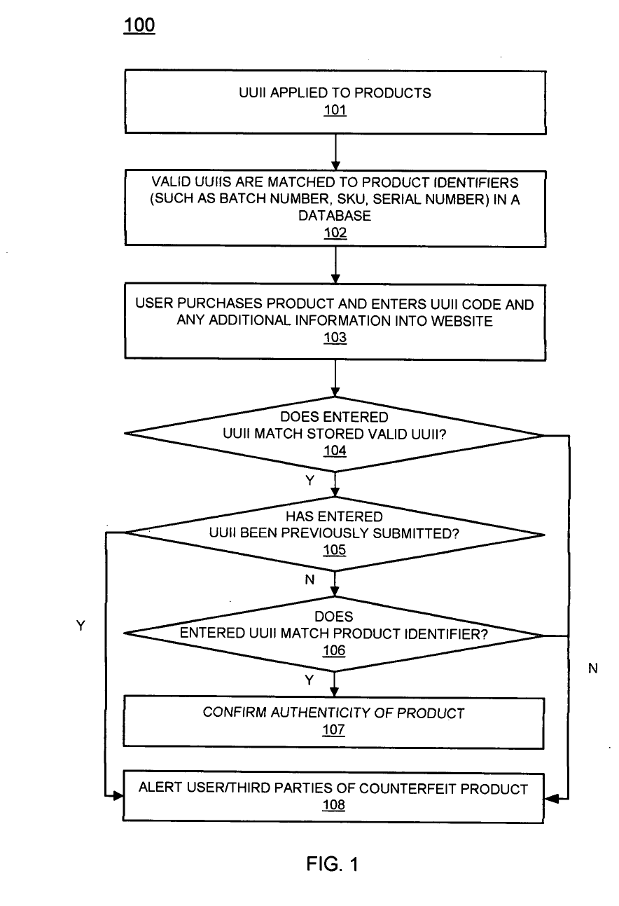

Referring now to FIG. 1, therein is disclosed an exemplary process 100 for using UUIIs to authenticate products. In performing the process 100, it should be readily apparent that the users and third party participants to the process 100 may communicate over a computer network, such as the Internet, and that each party may use any of a wide variety of well known computer systems that include processors, memory, inputs/outputs, communications ports, and appropriate hardware and software, such as operating instructions, applications, databases and processing instructions for accomplishing the functions described herein.

The process 100 begins when a party affixes a UUII code to an item of product (step 101), which is then released for sale to the general public. The UUII includes a date/time stamp or the like that uniquely identifies, via any of a variety of encoding techniques, the time the UUII was generated and/or applied to the product or its packaging. Valid UUIIs are stored in a database or the like (step 102) that matches an indication of the UUIIs, such as a batch, to an indication of the product, such as a product identifier (SKU, UPC, serial number) of the product to which it was applied for later confirmation when a UUII is submitted by a user for authentication of the product.

When members of the general public purchase the product, they come online to a website to determine whether the product is an authentic product (step 103). They do so by entering the unique code which was originally affixed to the product by the manufacturer of the product. They may also enter in another piece of information that may identify their general location, such as a zip (or other postal) code, a city name, a telephone area code, a store from which the product was purchased, or the like. This piece of information, combined with the validity of the unique code and the date and time at which the code was validated, serves as a unique instance of the product being authenticated, which in turn is used to determine the authenticity of the product as described later below.

Continuing with the process 100, the UUII received from the user in step 103 is compared to an indication of valid UUIIs (step 104). If the UUII is a valid UUII, the process 100 continues to step 105 below. Otherwise, if the UUII is not a valid UUII, the process 100, continues to step 108, in which the user and any appropriate third parties may be notified of a potentially counterfeit product.

From step 104, when the UUII is determined to be a valid UUII, it is next determined whether the UUII has been previously submitted for authentication by this or another user (step 105). If so, the UUII is considered invalid. This is because all UUIIs are unique codes that can never be re-used from product to product, and since a counterfeiter may attempt to copy UUIIs from other products and repeatedly use them, for example. In such case of a duplicate UUII submission, the process 100 continues to step 108, in which the user and any appropriate third parties may be notified of a potentially counterfeit product. Otherwise, if the UUII has not been submitted before for authentication, the process 100 continues to step 106 below.

Next, at step 106, any information entered by the user to identify the product (other than the UUII), such as SKU, UPC, serial number, batch code, or the like, is compared to the product code of the product to which the UUII was applied. Only when the UUII and product information as submitted by the user matches the stored UUII and product identifier information in the database, and only when the UUII has not been previously submitted, will the UUII be finally determined as valid (step 107). Otherwise, when the submitted product information does not match the stored product information, the process 100 continues to step 108, in which the user and any appropriate third parties may be notified of a potentially counterfeit product. After either steps 107 or 108 above, the process 100 ends.

It should be appreciated that at or after step 108, the process 100 may include requiring the user to submit further specific information so that the locations where counterfeited products were purchased may be readily identified. Other additional steps may likewise be taken, such as notification of manufacturers, distributors law enforcement, or any third party of the detection of counterfeited products.

By empowering millions of consumers to help police the prescription drug supply, the methods of authentication described herein offer a more effective overall solution. It dramatically lowers costs (from sales lost to counterfeit products) and liability (e.g., for pharmaceutical companies found responsible for the effects of a counterfeit drug), eliminates the cost of RFID tags and readers, reduces threat of lawsuits, and prevents loss of sales due to counterfeit drugs, which is currently estimated in the billions of dollars per year. The disclosed methods also reduce the liability and cost of insurance companies having to pay claims to patients who are not getting better because they are taking counterfeit medicine. Adaptation of the disclosed methods may even saves lives, by offering a reliable means by which fewer consumers will unwittingly take dangerous counterfeit drugs.

While the present disclosure mentions prescription drugs and pharmaceuticals in particular, it should be readily apparent that the methods herein may be applied to any of a wide variety of items and products, without limitation. While the present disclosure describes application of a UUII at one time, the UUII may be generated in parts separately, by separate parties and combined, for example, at a point of sale, after which the combined code is stored in a database. The use of UUIIs may be used in conjunction with RFIDs placed on products or as an alternative thereto. Various other equivalent steps may likewise be included in the disclosure provided herein.

In various embodiments, for increased security and safety, the UUIIs themselves may not be stored in a database as described in the foregoing. In such embodiments, the method 100 instead uses a decoding algorithm to reverse-calculate the information encoded into UUIIs received from users. Once this reverse calculation is done, then the decoded information is matched against stored uncoded information relating to each UUII (e.g., what product it was affixed to, the location the product was made, and any other information that may be encoded into the UUII).

Although the best methodologies have been particularly described in the foregoing disclosure, it is to be understood that such descriptions have been provided for purposes of illustration only, and that other variations both in form and in detail can be made thereupon by those skilled in the art without departing from the spirit and scope thereof, which is defined first and foremost by the appended claims.

What is claimed is:

1. A method for using a universally unique item identifier (UUII) for consumer marketing, the method comprising:
receiving, from a user, a UUII corresponding to a product, the UUII including an indication of a time of generation of the UUII;
determining a validity of the UUII; and
when the validity of the UUII is not confirmed, providing the user with an indication that the product is a counterfeit product.

2. The method of claim 1, wherein the UUII is received over the Internet at a web site hosting the online environment.

3. The method of claim 1, wherein the product is sold in the marketplace and has the UUII imprinted thereon.

4. The method of claim 1, wherein the product is a pharmaceutical product.

5. The method of claim 1, said receiving further comprising:
receiving an indication of a location of the user.

6. The method of claim 5, further comprising:
alerting a third party of the counterfeit product and the location of the user.

7. The method of claim 1, said determining further comprising:
comparing the UUII to a database of valid UUIIs.

8. The method of claim 7, said determining further comprising:
determining that the UUII received from the user is not valid when the UUII received from the user does not match a valid UUII from the database.

9. The method of claim 1, said determining further comprising:
determining that the UUII received from the user is not valid when the UUII received from the user has been previously entered by another user.

10. The method of claim 1, further comprising:
receiving a product identifier from the user, said determining further comprising determining that the UUII received from the user is not valid when the UUII received from the user does not correspond to the product identifier received from the user.

11. The method of claim 1, further comprising:
requesting further information from the user when the UUII is not valid.

12. An apparatus for using a universally unique item identifier (UUII) for consumer marketing, comprising:
an input for receiving, from a user, a UUII corresponding to a product, the UUII including an indication of a time of generation of the UUII;
memory for storing at least one of an algorithm and a database for use in confirming a validity of the UUII;
a processor for determining a validity of the UUII and, when the validity of the UUII is not confirmed, for providing the user with an indication that the product is a counterfeit product.

13. A method for identifying a counterfeit product, comprising:
receiving, from a user, a unique code associated with an individual product and a product identifier of the individual product;
determining whether the unique code received from the user corresponds to a stored valid code;
determining whether the product identifier received from the user corresponds to a stored product identifier for the stored valid code;
determining whether the unique code received from the user has been previously submitted for identification;
determining a validity of the unique code only when the unique code received from the user corresponds to the stored valid code, the product identifier received from the user corresponds to the stored product identifier, and the unique code received from the user has not been previously submitted; and
when the validity of the unique code is not confirmed, providing the user with an indication that the product is a counterfeit product.
